(19) United States
(12) Patent Application Publication
(10) Pub. No.: US 2002/0001593 A1
(43) Pub. Date: Jan. 3, 2002

(54) Hemolysin fusion proteins, their production and use

(76) Inventors: Peter O'Hanley, Washington, DC (US); Guy LaLonde, Woodside, CA (US)

Correspondence Address: Stephen B. Maebius, FOLEY & LARDNER, Suite 500, 3000 K Street, N.W., Washington, DC, 20007-5109, US

(21) Appl. No.: 09/833,063

(22) Filed: Apr. 12, 2001

Related U.S. Application Data
(60) Provisional application No. 60/196,492, filed on Apr. 12, 2000.

(51) Int. Cl. A61K 39/02; C12P 21/04; C12N 15/31; C07K 14/195

(52) U.S. Cl. 424/190.1; 424/85.1; 435/69.7; 435/325; 435/320.1; 530/350

(57) ABSTRACT

Novel hemolysin fusion proteins can be produced by inserting a foreign nucleotide sequence encoding an immunogenic peptide in a region of HlyA corresponding to the CnBr II through CnBr V region of HlyA.

### FIELD OF THE INVENTION

The present invention relates to hemolysin fusion proteins, their production, and use.

### BACKGROUND OF THE INVENTION

A hybrid plasmid (pSF4000) was constructed originally in 1980 by Falkow et al. and characterized (Nature, 294:665-667, 1981; Infection and Immunity, 42:178-186, 1983; Infection and Immunity, 43: 156-160, 1984). In brief, from Escherichia coli J96 strain, an 11.7 kb SalI restriction endonuclease DNA fragment encoding chromosomal hemolysin (containing hlyA, hlyB, hlyC, and hlyD genes) was ligated into pACYC184.

This plasmid was further evaluated by Welch et al during the late 1980s (1985-1990). A series of papers by Welch's laboratory demonstrated the following findings about α-hemolysin: (a) α-hemolysin activity is localized to a 7.5 kb portion of pSF4000, (b) α-hemolysin activity is not linked to the first 66 amino-terminal amino acids or the 194 carboxy-terminal amino acids, (c) glycine-rich repeats with the eight amino acid being aspartic acid are critical for hemolytic activity, (d) α-hemolysin is secreted by a novel C-terminal dependent mechanism and rendered biologically active by hlyC during secretion, and (e) substitution of hydrophobic residues with amino acids with polar side groups between residues 270 and 330 inhibits hemolytic activity by preventing apparently a membrane-spanning domain to insert (J. Bacteriology, 163:88-93, 1985; J Bacteriology, 163:94-105, 1985; J. Bacteriology, 170:1622-1630, 1988; Infection and Immunity, 58:822-827, 1990).

O'Hanley et al. extended the observation that a common protective α-hemolysin epitope of J96 hemolysin resides in a CnBr II fragment corresponding to residues 2-160 (Infection and Immunity, 58: 3029-3035, 1990; Infection and Immunity, 59:2089-2096, 1991; Infection and Immunity, 61:1091-1097, 1993). HlyA binds to a series murine monoclonal antibodies elicited to detergent treated 110 kDa protein that was purified from hemolytic WAF100 strain (Infection and Immunity, 58: 3029-3035, 1990). In brief, Mab 132 binds to CnBr II fragment; Mab 943 binds to CnBr V; and Mab 835 binds to CnBr VI.

### SUMMARY OF THE INVENTION

In one embodiment, the present invention relates to novel immunogenic hemolysin fusion proteins comprising at least one foreign amino acid sequence inserted into a deleted region of HlyA, wherein said deleted region reduces or eliminates hemolysin-mediated pore formation.

Another embodiment is a plasmid comprising DNA encoding the immunogenic hemolysin fusion proteins of the invention.

Another embodiment is a host cell transformed by the plasmid of the invention

A further embodiment is a process for producing immunogenic compositions of the invention comprising culturing a host cell transformed by a plasmid of the invention.

A further embodiment relates to tailored vaccines produced from such immunogenic compositions.

It is also an object of the invention to provide methods for treating or preventing diseases using tailored vaccines.

### DETAILED DESCRIPTION OF THE PREFERRED EMBODIMENT

In one embodiment, the present invention relates to novel immunogenic hemolysin fusion proteins comprising at least one foreign amino acid sequence inserted into a deleted region of HlyA, wherein said deleted region reduces or eliminates hemolysin-mediated pore formation. Preferably, the deleted region is the CnBr II through CnBr V region of HlyA. The foreign amino acid sequence to be inserted may be any sequence designed to achieve a desired immunogenic effect in a particular subject. The foreign amino acid sequence may be larger or smaller than the length of the deleted region of HlyA. Preferred foreign amino acid sequences for insertion into the deleted region are at least one of cholera B toxin peptide, heat-labile enterotoxin peptide, a pilin sequence, an HIV protective epitope, a flagellin sequence, or a cytokine sequence, such as an interleukin, particularly IL-10 and IL-4.

A further embodiment relates to tailored vaccines produced from such immunogenic compositions. Vaccines may be formulated according to known techniques using the immunogenic compositions of the invention. The invention further provides methods for treating or preventing diseases comprising administering to a subject in need thereof a vaccine of the invention. The subject is preferably a human.

Another embodiment is a plasmid comprising a nucleotide sequence encoding a hemolysin fusion protein of the invention. Preferably, a 950 base SmaI-SmaI deletion mutant of α-hemolysin in hlyA but with intact hlyB, hlyC, and hlyD genes from an Escherichia coli hemolysin operon are included in the plasmid. A preferred plasmid used to make the plasmid of the invention is pSF4000, which can be used to cause expression and secretion of hemolysin fusion proteins for vaccine purposes. A preferred deletion site within HlyA is the CnBr II through CnBr V region of HlyA. The site of deletion is used to insert foreign nucleotide sequences in order to produce a hemolysin-chimeric fusion protein. Any foreign nucleotide sequence may be used which encodes an amino acid sequence that is immunogenic in a desired context. Preferred foreign nucleotide sequences are cholera B toxin peptide, pilins, flagellins, and cytokines such as IL-10 and IL-4.

Preferably, SmaI leader sequences are incorporated at either termini of the foreign nucleotide sequence in proper 3′ to 5′ or 5′ to 3′ orientation. The ligation of the foreign genetic information at this site provides “carrier capacity” to render a small peptide immunogenic.

Another embodiment is a plasmid encoding hemolysin in which the deletion reduces or eliminates hemolysin-mediated pore formation. This plasmid is used as a starting plasmid to make a plasmid of the preceding embodiment in which a foreign nucleotide sequence is inserted in the deleted region.

Another embodiment is a host cell transformed with a plasmid encoding a hemolysin fusion protein of the invention. Preferably, the host cell is E. coli.

Another embodiment is a process of producing a hemolysin fusion protein of the invention comprising culturing a host cell transformed with a plasmid of the invention in a suitable medium and recovering the hemolysin fusion protein from the medium. Optionally, in a further step the process extends to formulating a vaccine from the hemolysin fusion protein.

Furthermore, construction of foreign antigens linked to other immunomodulatory substances (e.g., cholera toxin B, heat labile enterotoxin peptides, and interleukins/cytokines) inserted in the deletion region yields a chimeric fusion protein platform for tailored TH1 and TH2 vaccine purposes.

This hlyA deletion shuttle vector can be used to transform Escherichia coli strains for vaccine production using standard fermentation technology. Thereafter, the chimeric fusion protein can be purified by precipitation or affinity chromatography techniques.

The hlyA deletion shuttle vector can be incorporated into pYG58 derivatives and used to transform avirulent Salmonella typhimurium, qa-2 mutagenized Actinobacillus pleuropneumoniae strains, or other relevant live-bacterial vaccinal strains. These live vaccinal strains would continue to secrete the genetically designed chimeric fusion protein during their life cycle.

It has been demonstrated that a 950 base SmaI-SmaI deletion in pSF4000 that corresponds to bases within CnBr III into CnBrV (viz., in the potential membrane spanning domain) produces an inactive 85 kDa HlyA truncated product in an HB101 strain. The secreted product was bound by Mab 132 and Mab 835. It was not bound by Mab 943 suggesting that the deletion occurred distal to the second methionine residue but proximal to the third methionine in the intact α-hemolysin moiety. Also, based on the monoclonal antibody binding pattern, this deletion spanned into CnBrV but not distal to fifth methionine residue. Insertion of foreign antigens (e.g., cholera B toxin peptide, pilins, flagellins, cytokines such as IL-10 and IL-4) using SmaI leader sequences proximal and up-stream to the intended foreign protein insert can be incorporated and secreted as a chimeric hemolysin fusion proteins at this site.

Furthermore aroA mutant Salmonella typhimurium SL7207 strain and HB101 strain have been transformed by pYG58 containing the 950 base SmaI-SmaI deletion in pSF4000. The pYG58 is a derivative of pYG10, a plasmid extracted from Actinobacillus pleuropneumoniae 80-8141 (Gene 85:243-246, 1989). In brief, a 10.75 kb SalI fragment of pSF4000 that was further modified by deletion of the 950 base SmaI-SmaI deletion in hlyA of hemolysin operon was ligated to the single SalI site pYG10. This plasmid has been used for conjugation with a number of bacterial species. The transformed bacteria harboring the mutagenized hlyA have been demonstrated to be stable for up to 200 generations without the need for antibiotic selection. Furthermore, the salmonellae transformants have been stable in infected mice when isolated from liver and splenic tissues. The current list of bacterial species transformed by conjugation with this genetic shuttle includes: HB101 of Escherichia coli, aroA mutant Salmonella typhimurium strain SL7207, and Actinobacillus pleuropneumoniae strains with a defective 3-dehydroquinase enzyme gene (i.e., dhg). In brief, the dhg gene of Actinobacillus pleuropneumoniae codes for a 3-dehydroquinase enzyme that is equivalent to the aroD gene in Escherichia coli (Molecular Biology 11:273-280, 1994). If this gene is mutagenized, it will render these bacteria incapable of replication and growth similar to strains with aroA, aroD, or aroC blocks.

The invention has been described above with reference to specific examples. Further modifications and variations known to those of ordinary skill based on the description herein are contemplated to be within the invention.

The disclosures of all cited references are expressly incorporated herein to the same extent as if each was individually incorporated by reference.

What is claimed is:

1. An immunogenic hemolysin fusion protein comprising at least one foreign amino acid sequence inserted into a deleted region of HlyA, wherein said deleted region reduces or eliminates hemolysin-mediated pore formation.

2. The protein of claim 1, wherein the deleted region is the CnBr II through CnBr V region of HlyA.

3. The protein of claim 1, wherein the foreign amino acid sequence is at least one of cholera B toxin peptide, heat-labile enterotoxin peptide, a pilin sequence, an HIV protective epitope, a flagellin sequence, or a cytokine sequence.

4. A vaccine comprising the protein of claim 1 and a pharmaceutically acceptable carrier.

5. A plasmid comprising a nucleotide sequence encoding the protein of claim 1.

6. A host cell transformed by the plasmid of claim 5.

7. A process of producing an immunogenic hemolysin fusion protein, comprising culturing the host cell of claim 6 in a suitable medium, and recovering the immunogenic hemolysin fusion protein secreted by the host cell.
